(19) United States
(12) Patent Application Publication
(10) Pub. No.: US 2016/0326476 A1
(43) Pub. Date: Nov. 10, 2016

(54) MICROFLUIDIC BIOREACTOR WITH MODULAR DESIGN FOR SYNTHESIZING CELL METABOLITES, METHOD FOR USING SAME, AND USE THEREOF

(71) Applicant: KARLSRUHER INSTITUT FÜR TECHNOLOGIE, Karlsruhe (DE)

(72) Inventors: Jan Maisch, Ettlingen (DE); Ralf Ahrens, Karlsruhe (DE); Shukhrat Sobich, Karlsruhe (DE); Andreas Guber, Karlsruhe (DE); Kristina Kreppenhofer, Munich (DE); Peter Nick, Freiburg (DE)

(21) Appl. No.: 15/105,054

(22) PCT Filed: Dec. 17, 2014
(86) PCT No.: PCT/EP2014/003394

§ 371 Date: Jun. 16, 2016

(30) Foreign Application Priority Data
Dec. 20, 2013 (DE) 10 2013 114 634.1

(51) Int. Cl. C12M 3/06; C12M 1/00; C12M 3/00

(52) U.S. Cl. CPC C12M 23/16; C12M 23/44; C12M 29/04

(57) ABSTRACT

A microfluidic bioreactor includes at least two modules each having a cavity, each of which is divided by a membrane into a cell chamber for receiving cells and a material chamber through which a liquid solution comprising at least one additive and cell metabolites can flow, wherein each membrane has a reaction region in which the membrane is permeable at least in part to the solution comprising the at least one additive and the cell metabolites, and a fluid conducting system for the liquid solution comprising the at least one additive and the cell metabolites, which fluid conducting system connects the material chambers together at least one of in series or in parallel. Unidirectional flow through the fluid conducting system is ensured.

### CROSS REFERENCE TO RELATED APPLICATIONS

This application is a U.S. National Stage Application under 35 U.S.C. §371 of International Application No. PCT/EP2014/003394 filed on Dec. 17, 2014, and claims benefit to German Patent Application No. DE 10 2013 114 634.1 filed on Dec. 20, 2013. The International Application was published in German on Jun. 25, 2015 as WO 2015/090581 A1 under PCT Article 21(2).

### FIELD

The present invention relates to a microfluidic bioreactor having a modular design for obtaining cell metabolites, to a method for using said microfluidic bioreactor, and to the use thereof for obtaining cell metabolites.

### BACKGROUND

Microfluidics is a growing, dynamic field of research, because the integration of microfluidic structures, for example channels or reservoirs, into microsystems is of interest for many technical fields of application. According to Whitesides [1], those fields of application include in particular analytical chemistry, molecular biology and microelectronics. The microfluidic chips developed at the beginning of the 1990s originally for highly parallelized chemical analysis (capillary electrophoresis in chip format, [2, 3]) quickly found possible applications in modified form in other scientific disciplines, for example, molecular biology, and also in industry. The fundamental works by Whitesides (for example Soft Lithography, [1, 4]) in particular accelerated these developments significantly, since simple and rapid methods were developed which could be used even in non-specialized laboratories in order to produce microfluidic and nanofluidic chips and structures which are suitable in particular for biomedical applications.

Only a very small number of microfluidic systems for the cultivation of cells of plant origin are known. Ko et al. [5] describe a microfluidic system for the cultivation of plant cells from PDMS, in which protoplasts from green leaves of tobacco Nicotiana tabacum L. were cultivated for ten days. The presented chip has a cell culture chamber in the form of a channel, a microfilter, and an inlet and an outlet. The microfilter is arranged in the cell chamber, in the form of a channel, and serves as a retaining barrier for the cells situated in the cell chamber. Cell culture medium flows through the channel at a rate of 50-100 μl/min. The cell viability was confirmed qualitatively by a fluorescent vital stain, but was not quantified. Many dead cells are to be seen on the microscopic images that are presented.

Thiébaud et al. [6] show a PDMS microfluidics chip for the cell culture of animal cells having eight cell culture channels and eight inlet openings, wherein the cells adhere to the inside of the PDMS channel treated with laminine and are thereby retained in the channel when cell culture medium flows through the channel.

### SUMMARY

In an embodiment, the present invention provides a microfluidic bioreactor for obtaining cell metabolites. The microfluidic bioreactor includes at least two modules each having a cavity, each of which is divided by a membrane into a cell chamber for receiving cells and a material chamber through which a liquid solution comprising at least one additive and cell metabolites can flow, wherein each membrane has a reaction region in which the membrane is permeable at least in part to the solution comprising the at least one additive and the cell metabolites, and a fluid conducting system for the liquid solution comprising the at least one additive and the cell metabolites, which fluid conducting system connects the material chambers together at least one of in series or in parallel. The fluid conducting system is configured to provide unidirectional flow therethrough.

### BRIEF DESCRIPTION OF THE DRAWINGS

The present invention will be described in even greater detail below based on the exemplary figures. The invention is not limited to the exemplary embodiments. All features described and/or illustrated herein can be used alone or combined in different combinations in embodiments of the invention. The features and advantages of various embodiments of the present invention will become apparent by reading the following detailed description with reference to the attached drawings which illustrate the following:

FIG. 1 shows a schematic design of a microfluidic bioreactor according to an embodiment of the invention;

FIGS. 2a and 2b are exploded views of a module of the bioreactor according to embodiments of the invention;

FIG. 3 shows a schematic sequence of the method according to an embodiment of the invention for obtaining cell metabolites;

FIGS. 4a-4c show variants of the first unit of a module according to embodiments of the invention;

FIGS. 5a-5c show variants having parallel and combined parallel-linear coupling of the individual modules according to embodiments of the invention;

FIG. 6 shows the cell viability of the cells from a practical example;

FIG. 7 shows the determination of the mitotic index from a practical example; and

FIG. 8 shows cell-cell communication from a practical example.

### DETAILED DESCRIPTION

Embodiments of the present invention overcome certain limitations and disadvantages of the prior art. An embodiment of the present invention provides a microfluidic bioreactor which overcomes the technical difficulties encountered when plant cells are used for synthesis, that is to say for obtaining cell metabolites. Furthermore, a biotechnological method is to be proposed which allows cell metabolites, whose production requires a plurality of synthesis steps, to be obtained in a simple manner. A further object is the use of the microfluidic bioreactor according to the invention for obtaining cell metabolites.

Plant secondary metabolism produces many medicinally active components. These are formed in the plant only in specific cells and require the interaction of various tissues. Biotechnological production in batch cultures is therefore not feasible. Extraction from the plant is laborious and limited because the components are present in only a small number of cells. Moreover, many of these plants are endangered and rare.

A microfluidic bioreactor according to an embodiment of the invention includes a plurality of modules and is modeled on the tissue structure of cells, in which different cell types exist side by side in compartments and communicate with one another. The products of each cell line are made available to the next cell line as starting materials. The cells of the cell line in the last module in each case produce the desired end product, namely the cell metabolite dissolved in a liquid, from the products of the cells of the preceding cell lines. This coupling takes place in a modular manner—each module contains cells of one cell line in which a specific metabolic step is preferably upregulated by overexpression of the corresponding key enzyme. The product is then discharged from the cell via an exporter and then transported into the next module of the microfluidic bioreactor, where it serves as a substrate for the next module. The modules can be recombined in a modular manner so that a large number of metabolic branches is possible with a small number of modules. The individual cell lines are housed in separate flat cell chambers which are connected via a porous membrane to material chambers located therebelow, by means of which provision and material exchange are ensured. Each cell chamber has a filling system in order to fill it with the cells of the particular cell line. Each individual cell chamber, together with the associated material chamber, forms a module, and these modules can then be connected to one another as desired. It is thereby possible to produce in the through-flow valuable components which are limited in the natural plant system and cannot be produced abiotically on account of their chemical complexity. Owing to the modular principle, a large number of variants—including those which do not occur at all in nature—can be produced from only a small number of structural elements.

Throughout the text, the term “cells” includes not only natural and transgenic cells of animal or plant cell lines but also protoplasts, that is to say cells from which the cell wall has been removed by enzymatic digestion, yeasts, fungi and bacteria.

A microfluidic bioreactor according to an embodiment of the invention comprises at least two modules, the order of which can be chosen freely. Each module comprises a cavity which is divided by means of a membrane into a cell chamber for receiving cells and a material chamber through which a liquid solution comprising at least one additive and cell metabolites can flow. The cell chamber serves to receive natural or transgenic cells, in particular natural or transgenic plant cells or protoplasts. The cell chamber and its filling system form a first unit of any given module of the microfluidic bioreactor. The material chambers are connected to one another in series and/or in parallel by a fluid conducting system. The liquid solution comprising the at least one additive and the cell metabolites forms a fluidic circuit which flows through the material chambers unidirectionally. The material chamber and the associated fluid conducting system form a second unit of the microfluidic bioreactor.

Each membrane has a reaction region, the membrane being permeable at least in part to the solution comprising the at least one additive and the cell metabolites and allowing the at least one additive in the liquid solution of the reaction unit to come into contact with the cells in the cell chamber, that is to say there is the possibility of material exchange between the cell chamber and the material chamber at least in part at least in the reaction region of the membrane.

Material exchange means that, in addition to water, organic molecules and salts, what are referred to as additives pass from the material chamber into the cell chamber and/or vice versa either by diffusion or via natural or artificial transport systems by means of the fluidic circuit. As a result of the material exchange, the additives pass from the material chamber into the cell chamber, reactants always being able to migrate in both directions, that is to say from the cell chamber into the material chamber and from the material chamber into the cell chamber. In other embodiments, the reaction region of the membrane allows the material exchange of a plurality or all of the additives in both directions.

The material chambers of the at least two modules, through which flow can take place unidirectionally, are in liquid communication with one another via the fluid conducting system, that is to say the material chamber of the first module is coupled via the fluid conducting system to the material chambers of the second modules and the material chambers of any further modules that are present, in such a manner that the liquid flows first through the material chamber of the first module and then, in order, through the material chambers of the at least one further module.

The fluidic circuit of the material chambers of the at least two modules is connected to a pressure-generating device, preferably a pump, in particular peristaltic or syringe pumps, or to a suction unit, in such a manner that the liquid flows in succession through the material chambers of the at least two modules connected in series. The fluidic circuit of the material chamber of the first module of the bioreactor is fed by a liquid in a storage vessel or a system which continuously mixes the liquid. In a further embodiment, the pressure gradient is generated by a different height of the storage vessel compared with the module. The flow rate can be influenced by the applied pressure and depends on the rate of reaction of the individual synthesis steps and the receiving of the reactants in the cells of the cell lines used. The synthesis is carried out at a flow rate of from 1 to 1000 μl/min, preferably from 10 to 500 μl/min and particularly preferably from 25 to 150 μl/min.

In one embodiment, the modules are coupled linearly or in series, that is to say one module is arranged behind a further module and the fluidic circuit passes through all the modules. In a further embodiment, the modules are coupled in parallel, that is to say at least two mutually independent modules are coupled behind one module. The fluidic circuit is thereby divided into a plurality of streams. Each stream passes through one leg of these modules connected in parallel. The number of modules connected in parallel per stream is independent of one another. It is thereby possible to produce a plurality of products simultaneously from a precursor. In a particular embodiment, the modules are coupled linearly and in parallel, that is to say behind one module there are connected more than one mutually independent module, there being connected behind those modules a further module which is supplied by the two preceding, mutually independent modules. The fluidic circuit is thereby divided after one module into a plurality of streams, which then pass through the modules connected in parallel. After passing through the modules connected in parallel, the plurality of streams of the fluidic circuit are combined and together pass through the at least one downstream module. It is thus possible, for example, to obtain a plurality of different products from a first product, which enters the plurality of modules connected in parallel as the starting material. The plurality of different products are then conducted as starting materials into at least one common module, where they are converted by the cells into one product. This is advantageous especially if the products of the modules in question interfere with the yield of the other product in a lasting manner, that is to say if a reactant A is to be reacted to form product B and a reactant C is to be reacted to form product D, but the synthesis of D does not take place or takes place only insufficiently in the presence of B. The number of modules connected in parallel per stream is independent of one another and is determined by the synthesis route. The number of linear-parallel branching points is also determined by the synthesis route. It is thus also possible to establish a plurality of mutually independent branching points in one microfluidic bioreactor.

The additives used are selected from nutrients, growth regulators, immune defense substances, activators, inhibitors and elicitors, selected from HrpZ, flg22, resveratrol, or inducing or selective agents. The nutrients, selected from organic molecules, amino acids, fats, salts, carbohydrates, vitamins, macroelements, microelements or trace elements, are used to feed the cells in the individual cell chambers. Depending on the cell culture used, all the plant culture media known in the literature are used, a person skilled in the art selects the particular culture medium depending on the cell line, the synthesis to be performed and any genetic modification of the cell line. In addition to the standard media, nutrient media adapted to specific cell cultures can also be used (see Murashige et al. [7]).

“Reactant” is understood to mean the starting materials, or educts, for the particular synthesis step in the particular module. The products of each cell line in a module are made available as the reactant to the next cell line in the downstream module. The last cell line produces the desired product, namely the desired cell metabolite. The liquid solution comprising at least one additive of the fluidic circuit therefore comprises at least one reactant in order to start the synthesis. In a further embodiment, the reactant is a nutrient or an activator.

Plant hormones, growth regulators and other bioactive molecules that are known in the literature, but also temperature and light signals or electrical signals (see Namdeo [8]), are used as activators and inhibitors. Activators, selected from plant hormones, growth regulators and other bioactive molecules, such as indoleacetic acid, 1-naphthylacetic acid, 2,4-dichlorophenoxyacetic acid, jasmonic acid or abscisic acid, are used to initiate specific reactions in the individual cell lines. To that end, the activator in one embodiment is added directly into the fluidic circuit, that is to say the activator flows through all the material chambers of the various modules. Depending on the membrane used, the activators then migrate into the cell chambers of the individual modules and come into contact with the cells. The choice of membranes and cell lines in the individual modules is thus dependent on the action of the activators on the cell lines. In a further embodiment, the activator is added directly to the particular cell line, for example via the supply line, that is to say only the cells in that module come into contact with the activator. If further modules are connected behind that module, it is possible for subsequent cells to come into contact with excess activator in the solution of the fluidic circuit if the activator is able to pass through the membrane. In a preferred embodiment, the membrane is not permeable to the activator, that is to say the activator cannot pass through the membrane and remains in the cell chamber of the module to which it was added.

Inhibitors, selected from plant hormones, growth regulators and other bioactive molecules, such as 1-N-naphthylphthalamic acid, oryzalin, latrunculin or phalloidin, and temperature and light signals, are used to suppress specific reactions in the individual cell lines. To that end, the inhibitor is added directly to the fluidic circuit, that is to say the inhibitor flows through all the material chambers of the various modules. Depending on the membrane used, the inhibitors then migrate into the cell chambers of the individual modules and come into contact with the cells. The choice of membranes and cell lines in the individual modules thus depends on the action of the inhibitors on the cell lines. Alternatively, in a further embodiment, the inhibitor is added directly to the particular cell line, for example via the supply line, that is to say only the cells in that module come into contact with the inhibitor. If further modules are connected behind that module, it is possible for subsequent cells to come into contact with excess inhibitor in the solution of the fluidic circuit if the inhibitor is able to pass through the membrane. In a preferred embodiment, the membrane is not permeable to the inhibitor, that is to say the inhibitor cannot pass through the membrane and remains in the cell chamber of the module to which it was added.

If temperature or light signals or electrical signals are used as activators or inhibitors, they enter the system from outside, and the material of the microfluidic bioreactor must be chosen accordingly.

The cell chambers serve to receive natural or transgenic cells of plant or animal cell lines, protoplasts, yeasts, fungi or bacteria, preferably natural or transgenic plant cells or protoplasts. In one embodiment, the cell chamber can be opened so that the cells can be introduced into the cell chamber from outside, preferably in the form of a suspension. In a further embodiment, each cell chamber has a supply line and a discharge line, which allows cells, suspended in a liquid, preferably in a cell culture medium, to be introduced into the system. The discharge line allows the excess liquid volume to be discharged. In a further embodiment of the microfluidic bioreactor, a plurality of cell chambers are connected via a common supply line to a common supply vessel and have a common discharge line. The discharge line of the preceding module thereby constitutes the supply line of the following module. This embodiment allows a plurality of cell chambers to be filled with cells of the same cell line. The cells, suspended in a liquid, are thus flushed through a supply vessel into the cell chamber of one module and further into the cell chambers of the following modules. In a further embodiment, the cell chamber does not have a discharge line; excess liquid is transported away via the connecting line of the material chamber.

Plant cells do not require adhesion to a substrate for their growth. The cells initially float freely in the cell chamber and then settle on account of their specific weight. In a preferred embodiment, the module is so designed that the cells settle on the membrane from above due to gravity. The cell chamber is closed after the cells have been introduced. For the use of cell lines which require adhesion to the cell chamber, or the membrane, materials and coatings that promote adhesion are used. They are chosen from the known materials and coatings according to the cell lines used.

Since, owing to the modular construction of the bioreactor, all the cell chambers can be filled separately, emptied separately and can have a constant flow passing through them, and since the cells settle in the corresponding cell chambers, no special retaining or unloading devices are necessary. It is thus also possible to flush the chambers loaded with cells constantly, preferably with a small volume stream, without the cells being displaced.

In a further embodiment, at least one of the two lines, namely the supply line and the discharge line, is connected to a pressure-generating source so that the cells, suspended in a liquid, are pumped into the cell chamber and/or excess liquid is pumped out of the cell chamber or the material chamber. In a particularly preferred embodiment, the pressure can be adapted to the particular cell line, in order to avoid damaging the cells.

Flat geometries of the cell chamber are preferred, since the cells located furthest away from the membrane are supplied with the reactant from the material chamber only by diffusion. The height of the cell chamber, the distance between the membrane and the opposite, delimiting wall of the cell chamber, corresponds to the height of a plurality of layers of the cell lines to be used. The height of the cell chamber should preferably be no more than 20 times the longest extent of the cell line to be used, particularly preferably no more than 10 times the longest extent of the cell line to be used. The use of small chamber and channel systems in the microfluidic production method is necessary owing to the limiting diffusion paths between the cells. In one embodiment, the cell chamber is therefore from 0.01 mm to 5 mm, preferably from 0.05 mm to 2.5 mm and particularly preferably from 0.1 to 1 mm high. The material chamber has the same height or a different height to the cell chamber. In one embodiment, the cell chamber is from 0.01 mm to 5 mm, preferably from 0.05 mm to 2.5 mm and particularly preferably from 0.1 to 1 mm high. The maximum extent of the cell chamber and of the material chamber, that is to say the area over which the cell chamber and the material chamber are connected to the membrane, is from 10 mm² to 5000 mm², preferably from 50 mm² to 2500 mm² and particularly preferably from 100 mm² to 1000 mm². The Tillable volume of the cell chamber is from 10 μl to 5000 μl, preferably from 20 μl to 2000 μl and particularly preferably from 50 μl to 1000 μl.

In a preferred embodiment of the microfluidic bioreactor, the individual modules are brought into contact with one another by a plug-in system. The individual modules can thereby be separated from one another, that is to say the feed lines and discharge lines of the material chambers of the individual modules are provided with connectors which can be brought into contact with one another, by means of connecting pieces, in such a manner that the liquid solution comprising the at least one additive is conveyed in the fluid conducting system from the storage vessel via the material chamber of the first module into the material chamber of the second module into the material chambers of any further modules present. The advantage of a plug-in system is that modules already loaded with cells can be fitted together in the order of the synthesis steps and, after synthesis of a cell metabolite, the order of the cell lines connected in series in the individual cell chambers of the modules can be changed as desired, without having to load the cell chambers again. Assembly by means of a plug-in system is preferably carried out in such a manner that the supply lines are pushed into an appropriate receiver of the chamber unit, sealing being achieved by O-rings.

In a further embodiment, a plurality of modules of the microfluidic bioreactor are applied to a common carrier, so that they are not variably connected to one another by means of a plug-in system but are permanently in the same order. This embodiment is suitable especially for standard syntheses having a constant design.

In a preferred embodiment, the fluid conducting system of the material chambers has valves which can be shut off individually. It is thus possible to remove individual modules from the system or to change the order of the individual modules during operation, that is to say when the microfluidic bioreactor is filled with liquid.

All production processes, equipment and materials relating to the bioreactor must be as biocompatible and clean as possible. The microfluidic structures are made of plastics materials, that is to say polymers, glasses or metals, preferably of polymers, so that suitable methods, for example, molding (hot stamping, injection molding), direct cutting, 3D printing, casting, injection molding, etching, lithography or rapid prototyping, can be used for their production.

The microfluidic bioreactor is preferably produced from thermoplastic, biocompatible polymers. Both units are particularly preferably made of polycarbonate (PC), polymethyl methacrylate (PMMA), cyclic olefin copolymer (COC) or polydimethylsiloxane (PDMS), polystyrene (PS), polysulfone (PSU), polyethylene terephthalate (PET), polytetrafluoroethylene (PTFE), polypropylene (PP) or polyethylene (PE) or mixtures thereof.

The two units are connected to the membrane by standard methods selected from thermal bonding, adhesive bonding, compression or ultrasonic welding. Polymer materials are preferably thermally bonded, ultrasonically welded or adhesively bonded to membranes of the same polymer. In an embodiment in which the two units are made of a different polymer to the membrane that is used, or when a metal membrane is used, they are connected by ultrasonic welding or adhesive bonding. Glass or metal units are adhesively bonded to the membrane. In one embodiment, the two units are made of the same material. In a preferred embodiment, the material of the membrane is the same material as that of the two units.

In one embodiment, the two units are produced by means of the rapid prototyping method from epoxy resins, which are then connected to the membrane by means of adhesive bonding. Thermal bonding has limitations, since only materials that are also available as the membrane can be used, because benefits may be realized when the housing and the membrane are made of the same material. In a further embodiment, the two units are cast in PDMS and the parts are then pressed together with a porous membrane. PDMS is resilient and seals by pressing with fastening devices suitable therefor. In a further embodiment, the units are made of Foturan®, a type of glass which can be structured by means of optical lithography and then etched. The etching of glass is also possible with a structured covering layer. In both cases, optically non-transparent surfaces are obtained.

In one embodiment, the microfluidic bioreactor is produced at least in part, preferably at least in the reaction region, from a transparent material, either in order to observe the cell culture by means of a microscope or in order to couple in light signals. In a further embodiment, non-transparent or colored plastics materials are used if cell growth is to take place under specific lighting conditions.

In preferred embodiments of the microfluidic bioreactor, individual modules are provided with cold-generating or heat-generating devices in order to be able to control the reaction conditions in a flexible manner. Cooling or heating coils or Peltier elements are preferably used for that purpose.

The membrane used is permeable, that is to say it allows material exchange between the cell chamber and the material chamber. To that end, the membrane in one embodiment has pores. By choosing a suitable pore size, the size of the migrating molecules and cells can be limited, that is to say molecules or also cells that are larger than the chosen pore size are unable to pass through the membrane. The choice of pore size is also determined by the cell line used. The cell sizes differ greatly according to the cell culture used. The cells of the BY-2 tobacco cell line (Nicotiana tabacum L. cv Bright Yellow 2; see Maisch et al. [9]) have an average length of 55 μm and an average width of 35 μm. In other cell lines there are also substantially larger cells, such as in the tobacco cell line VBI-( ) (Nicotiana tabacum L. cv Virginia Bright Italia; see Campanoni et al. [10]), which become up to 150 μm long and 75 μm wide. In addition, cell cultures having substantially smaller cells are also used, for example, Arabidopsis thaliana (L. var. Landsberg, see Desikan et al. [11]) or rice cell suspension cultures (see Cao et al. [12]). The chosen pore size prevents the cells of the cell line used from passing from the cell chamber into the material chamber, because the pores of the membrane are smaller in diameter than the cell line used. The pore density, that is to say the number of pores per unit area of film, likewise depends on the cell line to be used. In the case of small pores, a high pore density is preferred. Films having large pores should have a lower pore density, in order on the one hand to avoid tears, and thus enlarged pores, upon application of the pressure with which the liquid is moved through the second liquid circuit, but on the other hand also in order to avoid enlarged pore diameters where pores are situated too close together.

The membrane used is preferably made of polymers. In a particularly preferred embodiment, ion-track etched membranes are used.

In a further, preferred embodiment, membranes of polymers which are semi-permeable are used, that is to say membranes which allow specific substances to pass only in specific directions. This makes it possible for only selected additives to pass from the second fluidic circuit into the cell chamber, and for only selected additives to pass from the cell chamber into the second fluidic circuit. In a further embodiment, the membrane is made of metal and has a microscreen structure. The various embodiments can be combined freely with one another.

In order to obtain cell metabolites using the microfluidic bioreactor according to embodiments of the invention, the cells are introduced into the cell chambers according to method step a). In one embodiment, this is effected by opening the cell chamber and introducing the cells into the cell chamber from outside. The cell chamber is then closed in a liquid-tight manner. In a further embodiment, the cells are introduced into the system via the supply line by being fed, while applying pressure, from a storage vessel via the supply line into the cell chamber. Then, according to method step b), a liquid stream of a liquid solution comprising at least one additive is applied in the fluid conducting system of the microfluidic bioreactor for synthesis of the at least one cell metabolite. The liquid solution comprising the at least one additive thereby passes through the membrane from the material chamber into the cell chamber. In the cell chamber, at least one cell metabolite is synthesized with the cells, and the liquid solution comprising the at least one additive and the at least one cell metabolite passes through the membrane back into the fluid conducting system.

The liquid solution in the fluidic circuit comprises at least one additive. In preferred embodiments, the synthesis steps in the individual chambers are influenced under the action of inhibitors and activators. To that end, those additives are either added to the liquid solution in the fluidic circuit or applied directly into the individual modules. Likewise, the cell lines are supplied either with nutrients which are added to the liquid solution in the fluidic circuit, or by applying the nutrients directly into the individual modules. Additives are added directly into the individual modules via the open cell chamber, the supply line, or additional lines which lead directly into the cell chamber.

The synthesis in the first module starts as soon as the reactant in the liquid solution of the fluidic circuit migrates from the material chamber of the first module into the cell chamber of the first module and is there converted by the cells into a product. That product is then released by the cells into the liquid solution of the fluidic circuit and fed via the fluid conducting system from the first module into the next module. The solution that reaches the second module then comprises unreacted residues of the original starting material, the product of the synthesis in the first module and optionally further additives. The product of the synthesis of the first module then passes via the membrane of the second module into the cell chamber of the second module and is there taken up by the cells and converted into a further product, namely the product of the synthesis in the second module. This process is repeated as often as there are modules connected in series in the microfluidic bioreactor. The product of the synthesis of the last module constitutes the total product of the synthesis, the cell metabolite. The cell metabolite can be removed from the liquid stream according to method step c). To that end, the last module is followed in one embodiment by a collecting vessel. In a further embodiment, the liquid solution of the second fluidic circuit comprising the cell metabolite is fed directly to at least one purification means selected from preparative or semi-preparative chromatography, electrophoresis, extraction, precipitation, filtration, sedimentation or evaporation. In a preferred embodiment, purification takes place directly from the liquid solution. To that end, the installations which perform the cleaning steps are supplied directly via the discharge line of the microfluidic bioreactor according to the invention. In a particularly advantageous embodiment, the microfluidic bioreactor is integrated directly into a lab-on-a-chip.

The cell lines used in the individual modules are identical or different cell lines which perform identical or different synthesis steps. The product of each individual synthesis step depends on the cell line used, the reactant, the reaction conditions, and the activators and inhibitors, as well as on the further additives which come into contact with the cells in that module. They are in each case chosen having regard to the synthesis step that is to be performed.

In order to be able to carry out light-sensitive reactions, individual modules of colored, light-deflecting materials can be used. In the case of photochemical reactions, modules are used that are made of transparent materials which allow the wavelength necessary in a particular case to pass through. Within the reaction chain, individual modules are heated or cooled via heat-generating or cold-generating devices, according to the requirements of the synthesis steps.

Using the microfluidic bioreactor, cell metabolites can be produced by combining a wide variety of different cell lines. Plant or animal cell lines of both natural and genetically modified origin are used. For the production of cell metabolites using the bioreactor according to embodiments of the invention, cells are genetically modified substantially in three ways: 1. regulating the genes coding for the key enzymes of the corresponding metabolic pathways, 2. influencing the secondary metabolism by introducing new genes, 3. modifying the secondary metabolism by downregulation or overexpression of specific pathway genes (see Yeomann et al. [13]).

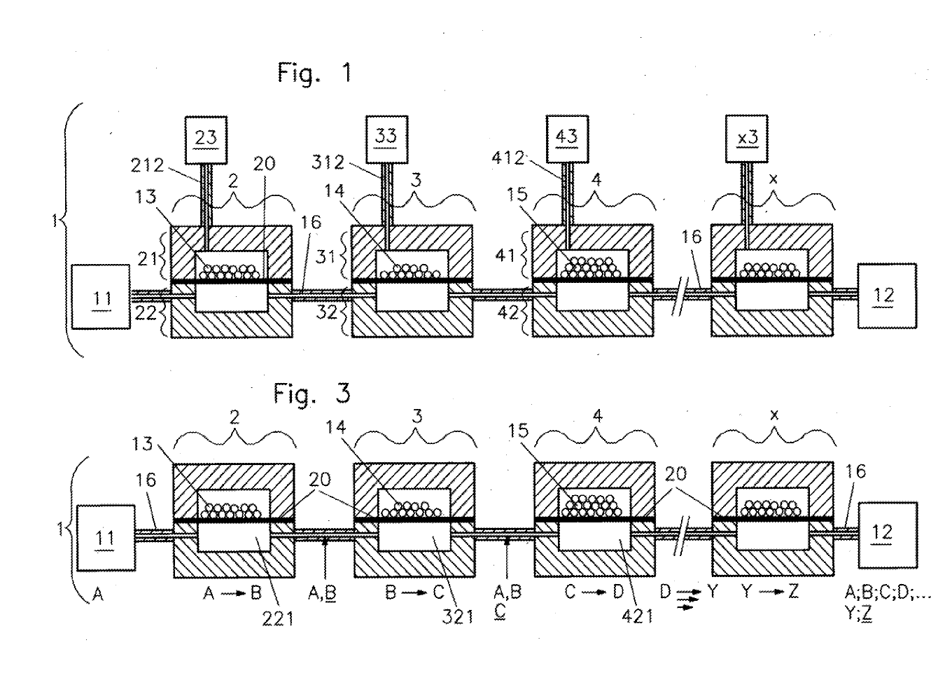

FIG. 1 shows the basic design of the microfluidic bioreactor (1) having a plurality of modules (2, 3, 4). The first module (2) consists of two units (21, 22). The first unit (21) constitutes the cell chamber, which is filled with the cells (13) from the supply vessel (23) via the supply line (212). The second module (3) consists of two units (31, 32). The first unit (31) of the second module (3) constitutes the cell chamber, which is filled with the cells (14) from the supply vessel (33) via the supply line (312). The further modules (4, X) likewise consist of two units (41, 42). Synthesis of the cell metabolite starts as soon as the liquid solution comprising the at least one additive passes from the storage vessel (11) via the fluid conducting system (16) into the material chamber of the second unit (22) of the first module (2) and from there into the further second units (32, 42) of the further modules (3, 4, X), and the reactant for synthesis of the cell metabolite migrates via the membranes (20) into the cell chambers and is there metabolized by the cells. The desired cell metabolite can then be removed from the collecting vessel (12).

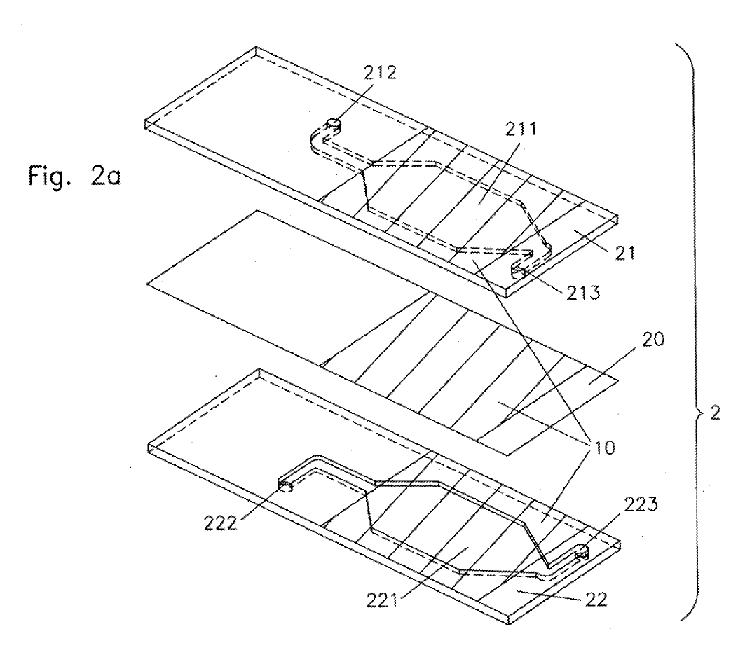

FIG. 2a is an exploded view of a model of a module (2) of the microfluidic bioreactor, having a first unit (21) having a cell chamber (211) and a second unit (22) having a material chamber (221) which is arranged in a form-fitting manner relative to the cell chamber (211) of the first unit (21, 31, 41), and a membrane (20) which is so introduced between the first unit (21) and the second unit (22) that it separates the cell chamber (211) from the material chamber (221) and which is permeable at least in part in the reaction region (10) for contacting the reactant in the liquid solution of the material chambers (221) with the cells in the cell chamber (21). The material chamber further has a feed line (222) and a connecting line (223) by means of which it is integrated into the fluid conducting system of the microfluidic bioreactor.

FIG. 2b is an exploded view of a model of a combination of modules of the microfluidic bioreactor (1), having three cell chambers (211, 311, 411) and three material chambers (221, 321, 421) and a membrane (20) which is so introduced between the three cell chambers (211, 311, 411) and the three material chambers (221, 321, 421) that it separates the cell chambers (211, 311, 411) from the material chambers (221, 321, 421) and which is permeable at least in part in the reaction region (10) for contacting the reactant in the liquid solution of the material chambers (221, 321, 421) with the cells in the cell chambers (211, 311, 411). The first cell chamber (211) is thereby situated in a form-fitting manner on the first material chamber (321). The second cell chamber (311) is thereby situated in a form-fitting manner on the second material chamber (321). The third cell chamber (411) is thereby situated in a form-fitting manner on the third material chamber (421). Furthermore, the material chambers are integrated into the fluid conducting system (16) of the microfluidic bioreactor. Each of the cell chambers (211, 311, 411) also has a supply line (212, 312, 412) and a discharge line (213, 313, 413).

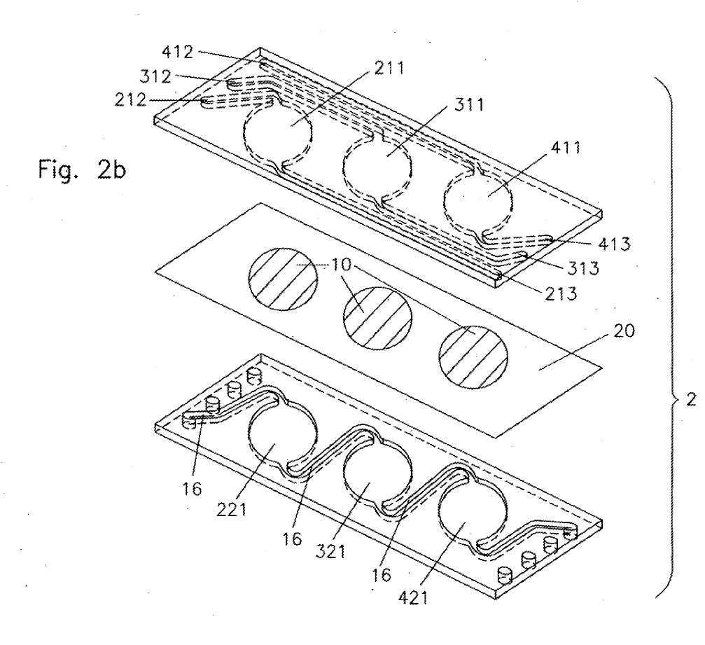

FIG. 3 shows the basic principle of the synthesis of a cell metabolite using the microfluidic bioreactor according to the invention. The liquid solution of the fluidic circuit of the fluid conducting system (16) flows from the storage vessel (11) through the material chamber (221) of the first module (2). The liquid solution from the storage vessel comprises at least one additive, namely the reactant A. As the solution flows through the module (2), the reactant A is conveyed from the material chamber (221) into the cell chamber (211), where it comes into contact with the cells (13). The cells (13) react A to form B and release B into the fluidic circuit of the fluid conducting system (16). The liquid solution flowing from the module (2) contains both excess reactant A and the newly produced product B. As the solution flows through the next module (3), the product B (now reactant B) is conveyed from the material chamber (321) into the cell chamber (311), where it comes into contact with the cells (14). The cells (14) react B to form C and release C into the fluidic circuit of the fluid conducting system (16). The liquid solution flowing from the module (3) contains both excess reactants A and B and the newly produced product C. As the solution flows through the next module (4), the product C (now reactant C) is conveyed from the material chamber (421) into the cell chamber (411), where it comes into contact with the cells (15). The cells (15) react C to form D and release D into the fluidic circuit of the fluid conducting system (16). The liquid solution flowing from the module (4) contains both excess reactants A, B and C and the newly produced product D. Depending on the number of modules, the whole process is repeated until the product Y, namely the reactant for the synthesis of the desired cell metabolite, has been produced and released into the liquid solution. As the solution flows through the last module (X), the product Y (now reactant Y) is conveyed from the material chamber into the cell chamber, where it comes into contact with the cells. The cells react Y to form the desired cell metabolite Z and release it into the fluidic circuit of the fluid conducting system (16). The liquid solution flowing from the module (X) contains both excess reactants A, B, C to Y and the newly produced product, the desired cell metabolite Z. The liquid solution is then transported further via the fluidic circuit of the fluid conducting system to a purification system or a collecting vessel (12).

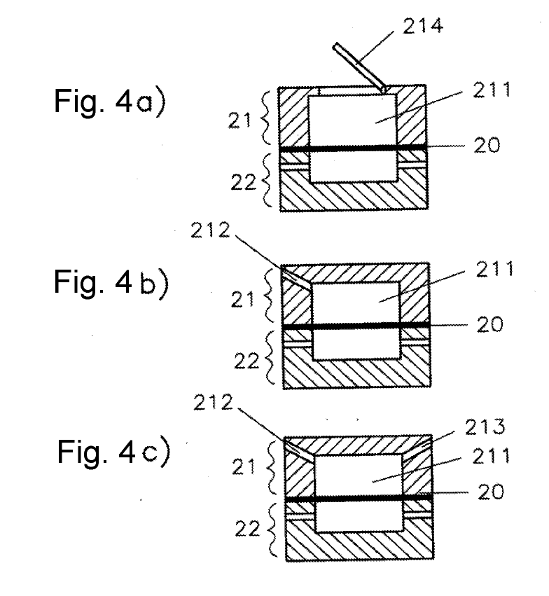

FIG. 4 shows, schematically, different designs of the first unit (21) of a module (2) of the microfluidic bioreactor (having the second unit (22) and the membrane (20)) a) shows filling of the cell chamber (211) by opening the cell chamber by means of a lid (214); b) shows filling via the cell chamber's own supply line (212), excess liquid being discharged via the material chamber; and c) shows filling via the cell chamber's own supply line (212). Discharge is carried out via an intrinsic discharge line (213).

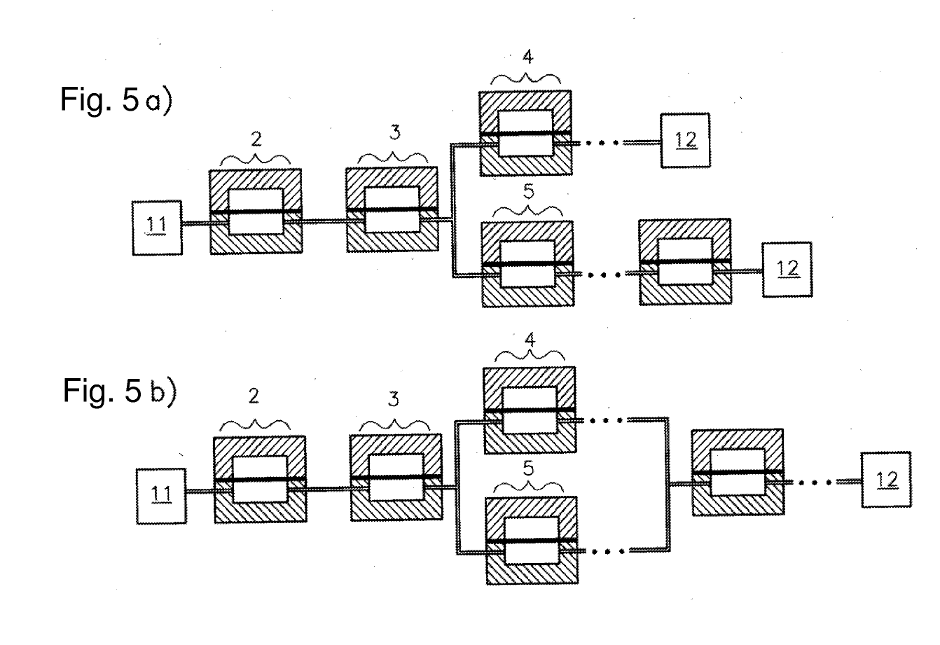

FIG. 5 shows designs having parallel and combined parallel-linear coupling of the individual modules (2, 3, 4, 5, 6) of the microfluidic bioreactor connected to a storage vessel (11) and a collecting vessel (12): a) shows parallel coupling for the synthesis of a plurality of different cell metabolites, b) shows linear-parallel coupling having one branching point for the synthesis of one cell metabolite, two different reactants being produced from one starting material during the synthesis thereof, and c) shows linear-parallel coupling having a plurality of branching points.

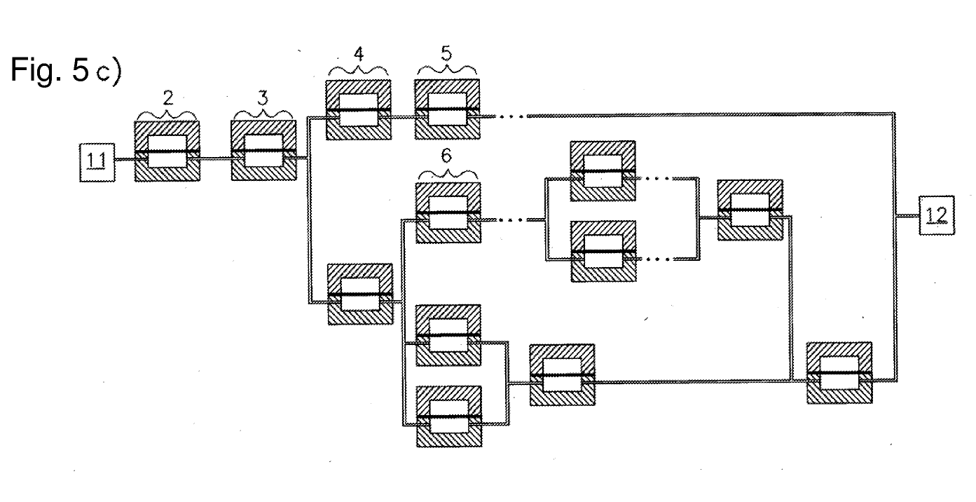

FIG. 6 shows the cell viability of the cells from practical example 4.

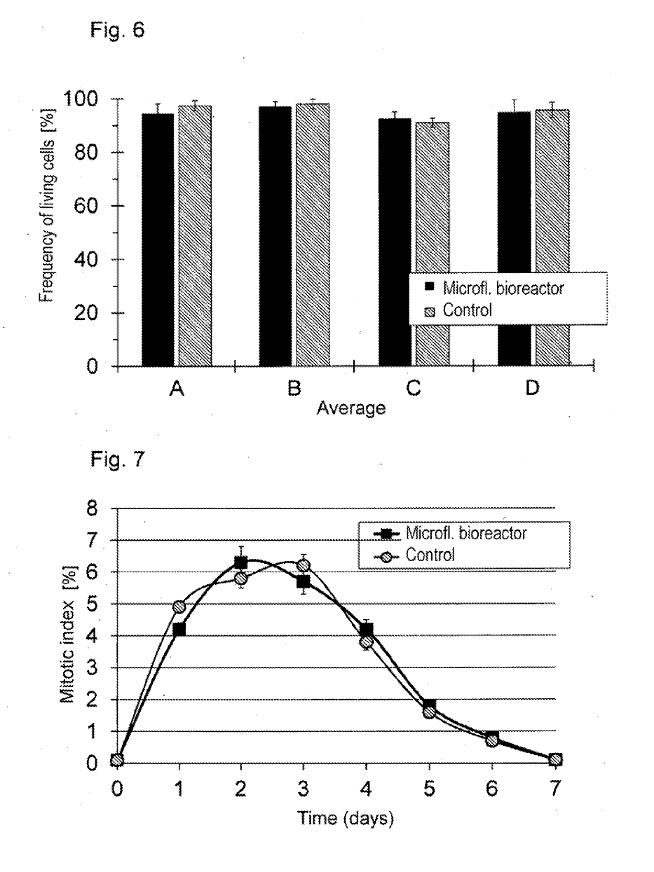

FIG. 7 shows the determination of the mitotic index from practical example 4. In the exponential growth phase (days 1 to 4), the mitotic index was between 4 and 6.5% in both batches.

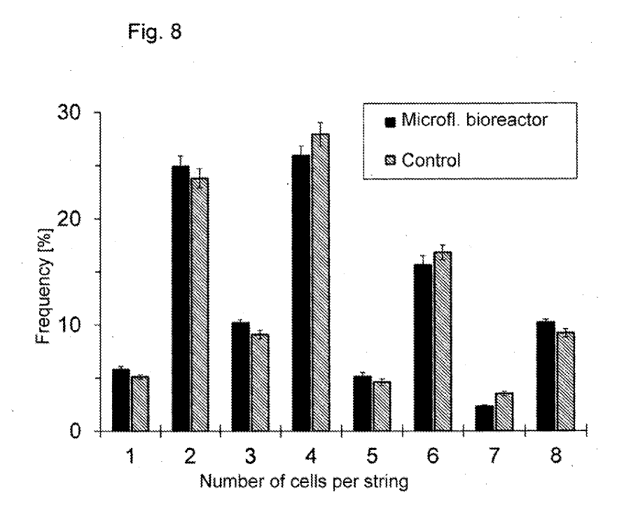

FIG. 8 shows the cell-cell communication and the coordinated growth from practical example 4. The characteristic maxima in respect of the frequency distribution of two-cell (25%), four-cell (27%) and six-cell strings (16%) of the 4-day-old culture were detectable in both test batches.

### Example 1

### Microfluidic Bioreactor Having a Hexagonal Chamber Geometry

Microfluidic bioreactor made of polycarbonate (PC) having a rectangular base area of 26 mm×76 mm and 2 mm thickness. The height per unit is 1 mm. The cell/material chambers are identical to one another, have a hexagonal shape and, with dimensions of 15 mm (width)×27.5 mm (length)×0.5 mm (height), provide a surface area of 300 mm². The fillable volume of the cell chamber is 150 μl. The chambers were produced by hot stamping.

The membrane used, namely a PC filter membrane having 0.4 μm pores, is “semi-porous”, that is to say it is porous only in the region of the cell/material chambers. The microfluidic bioreactor was operated at a flow rate of 75 μl/min.

### Example 2

### Microfluidic Bioreactor Having Three Elliptical Chambers on a Carrier without a Plug-in Connection

Microfluidic bioreactor made of polycarbonate (PC) having a rectangular base area of 26 mm×76 mm and 2 mm thickness. The cell/material chambers are identical to one another, have an elliptical shape and, with an ellipse radius of between 6 mm and 9 mm and a height of 0.5 mm, provide a surface area of 170 mm². The channels are 1.5 mm wide and 0.5 mm high. The fillable volume of the cell chamber is 85 μl. The structures are obtained by directly cutting into the PC base material. The membrane used is a porous PC filter membrane having 0.4 μm pores. The two units were connected to the membrane by ultrasonic welding. The microfluidic bioreactor was operated at a flow rate of 75 μl/min.

### Example 3

### Microfluidic Bioreactor Having One Elliptical Chamber

Microfluidic bioreactor made of polycarbonate (PC) having an elliptical base area of 10.5×26.8 mm and 2 mm thickness. The cell chamber and the material chamber are identical to one another, have an elliptical shape and, with ellipse radii of from 7.5 mm to 23.8 mm, provide a surface area of approximately 561 mm². The cell chamber is 0.5 mm high, the material chamber 1 mm. The channels are 1.5 mm wide and 0.5 mm high. The fillable volume of the cell chamber is 280 The membrane used is a PC filter membrane having 0.4 μm pores. The structures were obtained by directly cutting into the PC base material. The two units were connected to the membrane by ultrasonic welding. The microfluidic bioreactor was operated at a flow rate of 75 μl/min.

### Example 4

### Cell Culture of Tobacco BY2 Cells

The microfluidic bioreactor from example 1 was sterilized with 70% ethanol and then rinsed with sterile distilled water. The microfluidic bioreactor was then filled with a sterile MS medium (composition of the medium: [10]). The tobacco BY2 cells (Nicotiana tabacum L. cv Bright Yellow 2) were each removed from a suspension culture at different times after subcultivation (experiment A: 0 d, 50*10³ cells/ml; B: 2 d, 300*10³ cells/ml; C: 3 d, 850*10³ cells/ml) and introduced into the cell chamber by means of a sterile cannula via the supply line. The cells in the cell chamber settled on the membrane after about 10 minutes. MS medium flowed through the reaction chamber at a constant flow rate of 75 μl/min (peristaltic pump). Every 10 minutes, a sample was removed for NMR analysis from the MS medium leaving the material chamber. The cells were removed from the cell chamber after 72 and 96 hours and analyzed in respect of vitality, cell division and cell-cell communication (determined by standard methods [10]).

| | Loading: | Time in the microfluidic | Removal: |
|---|---|---|---|
| Experiment | Cell age (d) | bioreactor (h) | Cell age (d) |
| A | 0 | 96 | 4 |
| B | 2 | 72 | 5 |
| C | 3 | 96 | 7 |

| D | Average values of experiments A-C | |
|---|---|---|
| Control from | | 4, 5, 7 |
| suspension | | |
| culture | | |

It was possible to show that the cells cultivated in the microfluidic bioreactor exhibited the same properties in respect of vitality, cell division and cell-cell communication as the control cells, which grew under standard conditions in suspension culture. The diagrams shown in FIGS. 6 to 8 represent 3000 cells (vitality, mitotic index) or cell strings (frequency distribution, cell-cell communication) from in each case three independent experimental series. The error bars show standard errors. The survival rate of 4- (A), 5- (B) or 7- (C) day-old cells was over 95% in the case of both the cells cultivated in the bioreactor and the control cells from the suspension culture (FIG. 6). Determination of the mitotic index (FIG. 7) showed that the rate of division of the cells in the bioreactor was comparable with that of the control cells. In the exponential growth phase (days 1 to 4), the mitotic index was between 4 and 6.5% in both batches.

No differences were found between the two test batches in respect of cell-cell communication and coordinated growth either (FIG. 8). The characteristic maxima in respect of frequency distribution of two-cell (25%), four-cell (27%) and six-cell strings (16%) of the 4-day-old culture were detectable in both test batches.

In addition to the analysis of metabolic fluxes, it was also possible by means of NMR analysis to detect numerous substances which were released into the medium stream by the cells cultivated in the bioreactor, in concentrations of from 10 μm to 100 mM (for example glycolic acid, phosphoethanolamine, sarcosine, tartaric acid, taurine, trimethylamine oxide, trimethylamine).

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

### LIST OF REFERENCE NUMERALS

1 Microfluidic Bioreactor
2, 3, 4, 5, 6, X Module
10 Reaction Region
11 Storage Vessel
12 Collecting Vessel
13, 14, 15 Cells
16 Fluid Conducting System
20 Membrane
21, 31, 41 First Units
22, 32, 42 Second Units
23, 33, 43, X3 Supply Vessel
211, 311, 411 Cell Chamber
212, 312, 412 Supply Line
213, 313, 413 Discharge Lines
214 Lid
221, 321, 421 Material Chambers
222 Feed Lines
223 Connecting Lines

### LITERATURE

[1] Xia Y., Whitesides G. M.: Soft Lithography. Angew Chem, Int Ed Engl 1998, 37(5): 550-575.
[2] Harrison D. J., Manz A., Fan Z., Lüdi H, Widmer H. M.: Capillary Electrophoresis and Sample Injection Systems Integrated on a Planar Glass Chip, Anal. Chem. 1992, 64, p. 1926-1932.
[3] Manz A., Harrison D. J., Verpoorte E. M. J., Fettinger J. C., Paulus A., Lüdi A., Widmer H. M.: Planar chips technology for miniaturization and integration of separation techniques into monitoring systems: Capillary electrophoresis on a chip, J. Chromatogr. 1992, 593, p. 253-258.
[4] Whitesides G. M.: The origins and the future of microfluidics. Nature 2006, 442(7101): 368-373.
[5] Ko, J. M., Ju, J., Lee, S., Cha, H. C.: Tobacco protoplast culture in a polydimethylsiloxane-based microfluidic channel. Protoplasma, 2006. 227: p. 237-240.
[6] Thiébaud P., Lauer L., Knoll W., Offenhäusser A.: PDMS device for patterned application of microfluids to neuronal cells arranged by microcontact printing Biosensors and Bioelectronics, 2002. 17: p. 87-93.
[7] Murashige T., Skoog, F.: A revised medium for rapid growth and bio assays with tobacco tissue cultures. Physiologia Plantarum 1962. 15, 3: 473-497; Linsmaier E. M., Skoog F.: Organic growth factor requirements of tobacco tissue cultures. Physiologia Plantarum 1965. 18, 1: 100-127; Gamborg O. L., Miller R. A., Ojima K.: Nutrient requirement of suspension cultures of soybean root cells. Exp. Cell Res. 1968. 50, 1: 151-158; Smith R. H.: Plant tissue culture, Elsevier, Academic press, 2012
[8] Namdeo A. G.: Plant cell elicitation for production of secondary metabolites: a review. Phcog. Rev. 2007. 1, 1: 69-79; Santner A., Calderon-Villalobos L. I. A., Estelle M.: Plant hormones are versatile chemical regulators of plant growth. Nature Chemical Biology 2009. 5, 5: 301-307; Qiao F., Petrasek J., Nick P.: Light can rescue auxin-dependent synchrony of cell division in a tobacco cell line. J. Exp. Botany 2010. 61, 2: 503-510; Hicks G. R., Raikhel N. V.: Small molecules present large opportunities in plant biology. Annu. Rev. Plant Biol. 2012. 63: 261-282.
[9] Maisch J., Nick P.: Actin is involved in auxin-dependent patterning. Plant Physiol. 2007. 143: 1659-1704.
[10] Campanoni P., Blasius B., Nick P.: Auxin transport synchronizes the pattern of cell division in a tobacco cell line. Plant Physiol. 2003. 133: 1251-1260.
[11] Desikan R., Hancock J. T., Coffey M. J., Neill S. J.: Generation of active oxygen in elicited cells of Arabidopsis thaliana is mediated by a NADPH oxidase-like enzyme. FEBS letters 1996. 382: 213-217.
[12] Cao J., Duan X., McElroy D., Wu R.: Regeneration of herbicide resistant transgenic rice plants following microprojectile-mediated transformation of suspension culture cells. Plant Cell Reports 1992. 11: 586-591.
[13] Yeoman M. M., Yeoman C. L.: Manipulating secondary metabolism in cultured plant cells. New Phytol. 1996. 134: 553-569.

1: A microfluidic bioreactor for obtaining cell metabolites, comprising
at least two modules each having a cavity, each of which is divided by a membrane into a cell chamber for receiving cells and a material chamber through which a liquid solution comprising at least one additive and cell metabolites can flow, wherein each membrane has a reaction region in which the membrane is permeable at least in part to the solution comprising the at least one additive and the cell metabolites; and
a fluid conducting system for the liquid solution comprising the at least one additive and the cell metabolites, which fluid conducting system connects the material chambers together at least one of in series or in parallel,
the fluid conducting system being configured to provide unidirectional flow therethrough.

2: The microfluidic bioreactor according to claim 1, wherein the individual modules can be brought into contact with one another by a plug-in system.

3: The microfluidic bioreactor according to claim 1, wherein each cell chamber is connected via its own supply line to its own supply vessel and has its own discharge line.

4: The microfluidic bioreactor according to claim 1, wherein a plurality of the cell chambers are connected to one supply vessel.

5: The microfluidic bioreactor according to claim 1, wherein the material chambers of the individual modules are connected to the fluid conducting system by connecting lines which have valves which can be shut off individually.

6: A method for obtaining cell metabolites using a microfluidic bioreactor according to claim 1, the method comprising:
a) introducing cells into the cell chambers;
b) applying a liquid stream of a liquid solution comprising at least one additive in the fluid conducting system of the microfluidic bioreactor for the synthesis of the at least one cell metabolite, wherein the liquid solution comprising the at least one additive enters the cell chambers via the material chambers through the membranes, wherein at least one cell metabolite is synthesized in the cell chambers by the cells, and wherein the liquid solution comprising the at least one additive and the at least one cell metabolite is fed back into the fluid conducting system through the membranes via the material chambers; and
c) removing the at least one cell metabolite from the liquid stream.

7: The method for obtaining cell metabolites using a microfluidic bioreactor according to claim 6, wherein the introducing cells into the cell chambers comprises introducing the same cell line or different cell lines or partially different cell lines into the respective cell chambers of the modules.

8: The method for obtaining cell metabolites using a microfluidic bioreactor according to claim 6, wherein the introducing cells into the cell chambers comprises introducing the cells in a nutrient solution via respective supply lines and removing excess volume of the nutrient solution via respective discharge lines.

9: The method for obtaining cell metabolites using a microfluidic bioreactor according to claim 6, wherein the synthesis in b) is initiated by adding at least one activator to at least one cell line in at least one of the cell chambers.

10. (canceled)
